(19) United States
(12) Patent Application Publication
(10) Pub. No.: US 2025/0268581 A1
(43) Pub. Date: Aug. 28, 2025

(54) WEARABLE ULTRASOUND PATCHES

(71) Applicant: KONINKLIJKE PHILIPS N.V., EINDHOVEN (NL)

(72) Inventors: Jaap Roger Haartsen, Best (NL); Jeffrey Visser, Eindhoven (NL); Vincent Adrianus Henneken, Utrecht (NL); Johannes Nicolaas Huiberts, Eindhoven (NL); Roberto Buizza, Eindhoven (NL); Cornelis Petrus Hendriks, Eindhoven (NL); Rafael Wiemker, Hamburg (DE); Thomas Koehler, Hamburg (DE); Joerg Sabczynski, Hamburg (DE)

(21) Appl. No.: 19/058,068

(22) Filed: Feb. 20, 2025

Related U.S. Application Data
(60) Provisional application No. 63/556,428, filed on Feb. 22, 2024.

(51) Int. Cl. A61B 8/00; G16H 30/40

(52) U.S. Cl. CPC A61B 8/5284; A61B 8/4236; A61B 8/4254; A61B 8/463; A61B 8/5223; G16H 30/40; A61B 2560/0462; A61B 2562/0204; A61B 2562/0219

(57) ABSTRACT

A non-transitory computer readable medium stores instructions executable by at least one electronic processor to perform an ultrasound monitoring method. The ultrasound monitoring method includes receiving at least one ultrasound image of a target tissue of a patient (P) acquired using an ultrasound imaging probe positioned at a target position on the patient; determining at least one data acquisition and/or data processing parameter based on the at least one ultrasound image of the target tissue acquired using the ultrasound imaging probe; and monitoring the target tissue of the patient including acquiring tissue data of the patient using an ultrasound patch comprising at least one ultrasound transducer placed at the target position on the patient and using the at least one data acquisition and/or data processing parameter.

### CROSS-REFERENCE TO RELATED APPLICATIONS

This patent application claims the priority benefit under 35 U.S.C. § 119 (e) of U.S. Provisional Application No. 63/556,428, filed on Feb. 22, 2024, the contents of which are herein incorporated by reference.

### FIELD OF THE INVENTION

The invention relates to a method for placing ultrasound patches and a wearable ultrasound patch.

### BACKGROUND OF THE INVENTION

Point-of-care ultrasound (POCUS) using handheld ultrasound devices such as the Philips Lumify (available from Koninklijke, Philips N.V., Eindhoven, the Netherlands) is rapidly gaining popularity across a multitude of healthcare specialties. It is a portable, fast, real-time, non-invasive and safe (non-ionizing radiation) imaging technique performed by a physician at the bedside and is standard practice in obstetric, emergency and musculoskeletal medicine. However, POCUS also has some limitations. For example, it is operator dependent, both in terms of image interpretation and in terms of repeatability and reproducibility since probe placement is done manually. Also, since it is a handheld device, it is less suitable for applications where continuous or regular measurements are desired. In addition, because of the Covid-19 pandemic, the demand for solutions that require as little nurse-patient interaction as possible has risen. For this purpose, wearable ultrasound patches are emerging.

An ultrasound patch is an ultrasound transducer that is integrated within a patch, a wearable device that connects to the skin and monitors health parameters of a patient. Advantages of such an ultrasound patch is that it allows for continuous and repeatable measurements, and after placement, the caregiver workload and nurse-patient contact is limited. Ideally, these ultrasound patches are small, wireless and low-cost. However, this is at the expense of functionality. For example, continuous high-resolution imaging requires a large transducer area, complex on-board electronics, high power consumption and a high data transfer rate. For monitoring purposes, a full image is not always needed. For example, for tracking a vein diameter, bladder diameter or muscle thickness, once the ultrasound patch is placed at the right location, it is sufficient to scan along one or a couple of scanlines instead of scanning along many scanlines to reconstruct a full image. Thus, ultrasound patches used for monitoring may only have a minimum functionality and not always provide full ultrasound images. This enables the use of relatively cheap ultrasound patches, but comes at an increased difficulty of finding the correct place on the surface of a subject for these ultrasound patches.

In standard point-of-care ultrasound, the diaphragm thickness is assessed at the zone of apposition (ZA) during inspiration and expiration, using a linear high frequency transducer of 10-15 MHz. The zone of apposition is the chest wall area where the lower rib cage reaches the abdominal contents. The probe is positioned between the antero-axillary and mid-axillary lines, perpendicular to the chest wall. The hemi-diaphragm is identified beneath the intercostal muscles as a hypo-echogenic layer of muscle tissue located between two hyper-echogenic lines (the pleural line and the peritoneal line). Diaphragmatic thickening is assessed by the thickening fraction (TF), calculated as the percentage inspiratory increase in the diaphragm thickness (Tₑᵢ) relative to end-expiratory thickness (Tₑₑ) during tidal breathing according to Equation (1):

$$TFdi = \frac{{T}_{ei} - {T}_{ee}}{{T}_{ee}} * 100 \%$$

with Tₑᵢ the end-inspiratory thickness.

Diaphragm thickness and thickness fraction can be assessed using B-mode and M-mode imaging. In case of M-mode, first a 2D B-mode movie is recorded. From the images a single scan line is selected that intersects the diaphragm region of interest. Next, a time-motion image of that scan line is plotted from which the diaphragm thickness and thickness fraction can be determined.

Monitoring patches may only have one or a few scanlines which makes it challenging to determine the anatomical structure of interest in the patch data.

In addition, such monitoring patches are intended to be used for extraction of geometrical information from an anatomical structure such as a diaphragm thickness or a blood vessel diameter. However, in case the anatomical structure orientation is not aligned orthogonal to the patch scanline(s) the structure thickness will be overestimated.

The following discloses certain improvements.

### SUMMARY OF THE INVENTION

It is, inter alia, an object of the invention to provide a method for placement of ultrasound patches, and a patch that enables the placement method.

In some aspects, a non-transitory computer readable medium stores instructions executable by at least one electronic processor to perform an ultrasound monitoring method. The ultrasound monitoring method includes receiving at least one ultrasound image of a target tissue of a patient acquired using an ultrasound imaging probe positioned at a target position on the patient; determining at least one data acquisition and/or data processing parameter based on the at least one ultrasound image of the target tissue acquired using the ultrasound imaging probe; and monitoring the target tissue of the patient including acquiring tissue data of the patient using an ultrasound patch comprising at least one ultrasound transducer placed at the target position on the patient and using the at least one data acquisition and/or data processing parameter.

In other aspects, an ultrasound imaging method includes performing ultrasound imaging using an ultrasound imaging probe to identify a target position on a patient at which the ultrasound imaging probe acquires images of a diaphragm of the patient; placing an ultrasound patch comprising at least one ultrasound transducer at the target position on the patient; determining at least one data acquisition and/or data processing parameter based on at least one ultrasound image of the diaphragm acquired by the ultrasound imaging probe during the ultrasound imaging; and acquiring target tissue data of the target tissue of the patient using the ultrasound patch placed at the target position on the patient and using the at least one data acquisition and/or data processing parameter.

In other aspects, an ultrasound imaging system includes at least one electronic processor programmed to receive ultrasound images of a target tissue of a patient acquired using an ultrasound imaging probe positioned at a target position on the patient; determine at least one data acquisition and/or data processing parameter based on at least one ultrasound image of the target tissue acquired by the ultrasound imaging probe during the ultrasound imaging; and monitor the target tissue of the patient including acquiring tissue data of the target tissue of the patient using an ultrasound patch comprising at least one ultrasound transducer placed at the target position on the patient and using the at least one data acquisition and/or data processing parameter.

In an aspect of the invention, there is provided a method for placing a wearable ultrasound patch, the method comprising:
connecting a first ultrasound transducer array to a patch;
moving the combination of the first ultrasound transducer array and the patch over a surface of a subject to a target position;
connecting the patch with the subject at the target position;
separating the first ultrasound transducer array from the patch; and
connecting a second ultrasound transducer array to the patch.

In this manner, a second ultrasound transducer array for monitoring purposes (that does not produce ultrasound images suitable for placement) can be placed without sacrificing on the target position accuracy. For example, a physician can choose the ultrasound transducer of their choice as the first ultrasound transducer array (e.g., Philips Lumify), connect a patch to this transducer, find the target position, connect the patch to the subject, and then replace the first ultrasound transducer array by a second ultrasound transducer array. However, as described in more detail below, embodiments of the invention may provide additional and/or alternative advantages.

According to an embodiment, the step of connecting the first ultrasound transducer array to the patch comprises:
connecting the first ultrasound transducer array to an adapter, and connecting the adapter to the patch; or
connecting the adapter to the patch, and connecting the first ultrasound transducer array to the adapter.

In some instances, the ultrasound transducer array of choice of the physician may not fit well or easily into the patch. In such cases, an adapter may be used to bridge size and/or shape differences between the patch and first ultrasound transducer array.

According to an embodiment, the step of moving the combination of the first ultrasound transducer array and the patch over a surface of a subject to a target position may further comprise:
generating ultrasound images with the first ultrasound transducer array; and
displaying the generated ultrasound images on a display.

In this manner the physician can visually, based on ultrasound images on a display, find the target position.

According to an embodiment, the first ultrasound transducer array (i.e., a probe) is part of an ultrasound imaging probe configured to be held by a user. For example, any ultrasound transducer array on the market such as any cart-based probes or the Philips Lumify probe.

According to an embodiment, the ultrasound probe is a hand-held ultrasound probe, such as, for example, the Philips Lumify probe.

According to an embodiment, the second ultrasound transducer array is part of an ultrasound probe configured to be used in a wearable ultrasound patch. For example, the second ultrasound transducer array is part of a probe designed to be wearable on the surface of a subject. In other words, the design is small and minimalistic so as to occupy little space and not pose hindrances to the subject wearing the second ultrasound transducer array patch combination. The combination of second ultrasound transducer array and patch is commonly known as wearable ultrasound patch.

According to an embodiment, the method may further comprise:
determining a first ultrasound signal parameter for generating an ultrasound image at the target location with the first ultrasound transducer array (310), and
determining a second ultrasound signal parameter for generating an ultrasound image at the target location with the second ultrasound transducer array (320), wherein the second ultrasound signal parameter is based on the first ultrasound signal parameter.

In this manner, the ultrasound signal parameter (i.e., frequency, propagation speed, wavelength, amplitude, focal position, time gain settings, power and/or intensity etc.) used by the first ultrasound transducer array may be conveyed to the second ultrasound transducer array, such that the ultrasound signal parameter only needs to be set once. Thus, the computing and/or manual labor to set the ultrasound signal parameters to monitor a particular feature within a subject from the target location is minimized. In an example, the ultrasound signal parameter used during the step of “moving the combination of the first transducer array and the patch over a surface of a subject to a target position” may be conveyed to the second ultrasound transducer array.

According to some embodiments, the method further comprises:
inserting a gel-pad into the patch, or
connecting a first gel-pad to the first ultrasound transducer array and connecting a second gel-pad to the second ultrasound transducer array.

According to an aspect of the invention there is provided a patch for a wearable ultrasound transducer, the patch comprising:
a first connection mechanism with a surface of a subject;
a second connection mechanism with a first ultrasound transducer array; and
a third connection mechanism with a second ultrasound transducer array.

The above patch thus enables the connection of a first ultrasound transducer array which may be contained in a typical ultrasound probe (e.g., Philips Lumify) and of a second ultrasound transducer array that may be within a probe designed to be worn by a subject. In this manner, enabling the method of placing a wearable ultrasound patch as described above.

According to an embodiment, the second connection mechanism and the third connection mechanism are the same.

According to an embodiment, the first connection mechanism comprises any one of:
an adhesive, wherein the adhesive connects the patch with the surface of the subject.
a vacuum, wherein a vacuum is formed between the patch and the surface of the subject.

According to an embodiment, the second and/or third connection mechanisms comprise any one of:
a click-based system configured to interlock the patch with the first ultrasound transducer array and/or the second ultrasound transducer array;
a pressure-based system, wherein a force pushes the first ultrasound transducer array and/or the second ultrasound transducer array onto the patch;
an adhesion-based system, wherein an adhesive connects the patch with the first ultrasound transducer array and/or the second ultrasound transducer array; and
a magnetic system, wherein a magnet connects the patch with the first ultrasound transducer array and/or the second ultrasound transducer array.

According to an embodiment, the second and/or third connection mechanisms further comprise:
an adapter configured to bridge size and/or shape differences between the patch and the first ultrasound transducer array and/or the second ultrasound transducer array.

According to an embodiment, the patch may further comprise a cut-out or region of transparent or translucent material (where transparent and translucent refer to properties of the material with respect to ultrasound), through which the first transducer array and second transducer array can send acoustic waves into the subject and receive the reflections of the acoustic waves from the subject.

According to an embodiment, the patch may further comprise a gel pad holder configured to hold a gel pad, wherein the gel pad is configured to be placed in the cut-out or region of transparent or translucent material:
between the surface of the subject and the first ultrasound transducer array and/or
between the surface of the subject and the second ultrasound transducer array.

These and other aspects of the invention will be apparent from and elucidated with reference to the embodiments described hereinafter.

One advantage resides in determining a location of a target tissue in imaging data.

Another advantage resides in determining accurately estimating a thickness of a diaphragm in ultrasound imaging data.

Another advantage resides in determining a placement of an ultrasound patch to acquire images of a diaphragm of a patient.

Another advantage resides in determining a thickness of a diaphragm for a patient undergoing mechanical ventilation therapy.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 is an exemplary flow chart of a method according to an embodiment of the invention.

FIGS. 2a-2e show exemplary locations for wearable ultrasound patch placements in lung ultrasound monitoring.

FIGS. 3a-3f display graphically the method of FIG. 1 according to an embodiment of the present invention.

FIG. 4a shows a patch according to an embodiment of the invention.

FIG. 4b shows an adapter for use in an embodiment of the invention.

FIG. 4c shows a minimum functionality ultrasound probe for use in an embodiment of the invention.

FIG. 5 displays a typical ultrasound system for use in an embodiment of the invention.

FIG. 6 shows an ultrasound imaging system in accordance with the present disclosure.

FIG. 7 show an example flow chart of operations suitably performed by the system of FIG. 6.

FIGS. 8-12 diagrammatically show an ultrasound image acquired by the ultrasound imaging system of FIG. 6 with one or more superimposed patch scanline markers.

### DESCRIPTION OF EMBODIMENTS

The invention will be described herein with reference to the Figures. It should be understood that the description and specific examples provided, while indicating exemplary embodiments, are intended for purposes of illustration only, and not intended to limit the scope of the invention. It should also be understood that the figures are mere schematics and not drawn to scale. It should also be understood that the same reference numerals are used throughout the Figures to indicate the same or similar parts.

The invention provides a method and devices for placement of a wearable ultrasound patch.

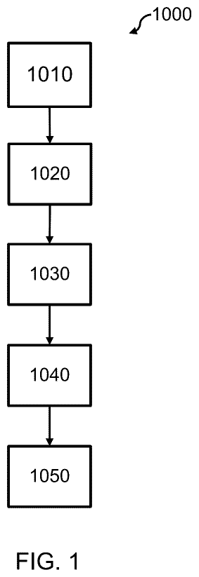

FIG. 1 displays a schematic of a method 1000 according to an embodiment of the present invention.

The method 1000 according to exemplary FIG. 1 describes the placement of a wearable ultrasound patch comprising the steps of:
i) Connecting 1010 a patch with a first ultrasound transducer array. In this example, the patch may be a device suitable for placement on a surface of a subject. For example, an adhesive device and/or a vacuum device as described further below. The first ultrasound transducer array may be an ultrasound transducer array of an ultrasound probe such as the Philips Lumify. The connection between the patch and the first ultrasound transducer array may further be enabled by any means, e.g.,
a click-based system configured to interlock the patch with the first ultrasound transducer and/or the second ultrasound transducer;
a pressure-based system, wherein a force pushes the first ultrasound transducer and/or the second ultrasound onto the patch;
an adhesion-based system, wherein an adhesive connects the patch with the first ultrasound transducer and/or the second ultrasound transducer; and
a magnetic system, wherein at least one magnet connects the patch with the first ultrasound transducer and/or the second ultrasound transducer.

The step 1010 of connecting the patch with the first ultrasound transducer array may optionally further comprise:
connecting the first ultrasound transducer array to an adapter, and connecting the adapter to the patch; or
connecting the adapter to the patch, and connecting the first ultrasound transducer array to the adapter.

In this manner, the patch may be designed without or with limited prior knowledge of the form factor and/or design of the first ultrasound transducer array. The adapter may thus enable a variety of first ultrasound transducer arrays, for example 1D (one-dimensional), 1.XD, 2D and/or 3D ultrasound transducer arrays, to be connected to the patch, and to be used according to the method 1000.
ii) Moving 1020 the combination of the first ultrasound transducer array and the patch over a surface of a subject to a target position. In this step, a physician may thus scan into the body of a subject to determine a target position for monitoring of a particular feature of the subject as discussed in more detail below. In particular, the physician may visualize the ultrasound data in an ultrasound image on a display in order to determine a target position for a monitoring activity. In other examples, an algorithm may indicate the target position based on, for example identified features in a feature detection assessment. In an example, the physician may adjust ultrasound signal parameters emitted from the first ultrasound transducer including but not limited to: the acoustic wave frequency, propagation speed, wavelength, amplitude, focal depth, time gain settings, power and/or intensity, to determine a target position for monitoring a particular feature or object within the subject. In an example, the ultrasound signal parameters emitted from the first ultrasound transducer may be adjusted or set automatically (e.g., by a machine learning module in response to an imaging procedure or based on detected features).
iii) Connecting 1030 the patch to the surface of the subject at the target position. Once a suitable target position is determined, the patch is connected to the surface of the subject. This connection may be done via an adhesive similar to tape or plasters, for example, the physician may remove an adhesive protective layer below the patch and glue the patch onto the surface of the subject, or via any other means. For example, vacuum may be used to connect the patch to the surface of the subject.
iv) Separating or disconnecting 1040 the first ultrasound transducer array from the patch. The first ultrasound transducer array may be separated by pulling the first ultrasound transducer array out, or by operating some mechanical, electronical or magnetic connection mechanism that releases the first ultrasound transducer array from the patch and allows for its retrieval.
v) Connecting 1050 a second ultrasound transducer array to the patch. The connection mechanism may be of any kind, for example, a click-based system, an adhesive, a magnetic system, etc.

Having determined a suitable target position, the first ultrasound transducer array is disconnected from the patch and a second ultrasound transducer array is inserted. In this manner, the first ultrasound transducer array used for finding the target position is replaced by a second ultrasound transducer array suitable for monitoring, and not necessarily including the functionality and/or capability of enabling a physician or an algorithm to determine the target location. Both ultrasound transducer arrays may be suitable for determining the target position and for monitoring.

The first ultrasound transducer array and second ultrasound transducer array may comprise any ultrasound transducer technology including but not limited to:
a piezoelectric transducer technology;
a capacitive micromachined ultrasonic transducer technology.

Connecting in the sense of steps 1010 and 1050 comprises enabling scanning of a subject by the first ultrasound transducer array and the second ultrasound transducer array through the patch. In some examples, the patch thus comprises a cut-out through which the first and second ultrasound transducer arrays may send acoustic signals (soundwaves) into the body of the subject. In other examples the patch comprises a translucent or transparent region through which the acoustic signals may be transmitted.

In order to convey the acoustic energy (soundwaves) from the first ultrasound transducer array and/or the second ultrasound transducer array into the surface of the subject with minimal reflection and refraction, a connecting contact medium may be necessary. For this purpose, typically ultrasound gel is used. However, in wearable ultrasound patches, ultrasound gel may not be convenient since it may hinder the attachment of the patch onto the surface of the subject. As a result, gel-pads may be used that may be attached directly to the ultrasound transducer array or inserted into the patch so as to be between the ultrasound transducer array and the surface of the subject. By inserting a gel-pad into the patch, the ultrasound data quality may thus be improved. In some examples, the patch may thus further comprise a region and/or cut-out for a gel-pad. In accordance, the method 1000 may further comprise a step of connecting a gel-pad with the patch or the first and second ultrasound transducer arrays. This optional step of placing the gel pad may be done at any stage, for example before connecting the first and/or second ultrasound transducer array to the patch or thereafter.

The gel-pad according to the preceding paragraph may be a solid ultrasound gel-pad.

It is obvious to a person skilled in the art that the steps of method 1000 may be carried out in a different order as presented here. For example, the patch and the first ultrasound transducer may be connected during the examination. The physician may briefly remove the first ultrasound transducer array from the surface of the subject to attach the patch and then re-place the first ultrasound transducer array-patch combination on the surface of the subject.

According to method 1000, an operator (e.g., a physician, a sonographer, a radiologist or any other person operating an ultrasound transducer and/or placing an ultrasound patch) may thus chose an ultrasound transducer array of their liking to search for a target position on a surface of a subject (e.g., patient, person or any object that is being examined or scanned), wherein the target position allows for monitoring a particular feature the physician wishes to have monitored. In particular, the operator may use a conventional ultrasound probe which comprises the first ultrasound transducer array, and visualize ultrasound images on a display such as an ultrasound cart, phone, tablet or any other monitor to aid in the determination of the target position. Once a target position is found, the operator then connects the patch to the surface of the subject. In some examples, the operator may again verify the target position before removing the first ultrasound transducer array. After removal of the first ultrasound transducer array, the operator inserts a second ultrasound transducer array. The second transducer array may be intended for monitoring purposes containing considerably less functionality than the first transducer array. In some examples, the second ultrasound transducer array is a minimum functionality product, capable only of scanning along a few scanlines and incapable of generating complete ultrasound images. This second ultrasound transducer array may however be capable of monitoring particular features of interest for an extended period of time, while not being connected to any external device or power supply. In other words, the patch and second ultrasound transducer array combination may be considered a wearable ultrasound patch. In other examples, the second ultrasound transducer array may comprise similar or the same functionality as the first ultrasound transducer array. In other examples the first ultrasound transducer array and second ultrasound transducer array may differ in size and/or shape.

In an example, the method may further comprise
determining a first ultrasound signal parameter for generating an ultrasound image at the target location with the first ultrasound transducer array, and
determining a second ultrasound signal parameter for generating an ultrasound image at the target location with the second ultrasound transducer array, wherein the second ultrasound signal parameter is based on the first ultrasound signal parameter.

In other words, the ultrasound signal parameters of the first ultrasound transducer array (e.g., frequency, propagation speed, wavelength, amplitude, power and/or intensity etc.) may be conveyed to the second ultrasound transducer array. For example, the optimal ultrasound signal parameters of the first ultrasound transducer array to visualize a particular feature inside the subject from the target position may be conveyed to the second ultrasound transducer array, so that the second ultrasound transducer array can optimally monitor said feature from the target position. The step of conveying the ultrasound signal parameters may be carried out by the operator or may happen automatically, for example if both ultrasound transducer arrays, the first and second ultrasound transducer arrays, are connected to the same computing device. In an example, the ultrasound signal parameters of the first ultrasound transducer array may be set by a physician. In an example, the ultrasound signal parameters of the first ultrasound transducer array may be set automatically by machine learning or artificial intelligence. In an example, the operator may overwrite and/or correct the ultrasound signal parameters received by the second ultrasound transducer array from the first ultrasound transducer array.

In an example, the ultrasound signal parameters of the second ultrasound transducer array may be set by a physician. In an example, the ultrasound signal parameters of the second transducer array may be set automatically by machine learning or artificial intelligence. In an example, the ultrasound signal parameters of the second ultrasound transducer array may deviate by a given (i.e., pre-defined) amount from the ultrasound signal parameter of the first ultrasound transducer array, for example to account for a different number of acoustic elements between the second and the first ultrasound transducer arrays.

Information gathered by the second ultrasound transducer array regarding the monitoring may be stored on local memory, or may be transmitted wiredly or wirelessly to an external memory. The transmission of the information may further take place during the monitoring examination or thereafter.

After the monitoring is complete, the second ultrasound transducer and patch combination may be disconnected from the surface of the subject.

In some examples, the patch is disposable.

In some examples, the first ultrasound transducer array and/or the second transducer array are re-usable.

In some examples the gel-pad is disposable.

The first and/or the second ultrasound transducer array in the meaning above may be used for any of the following applications:

Diaphragmatic ultrasound monitoring, e.g., continuous atrophy detection, diaphragm dysfunction detection, weaning prediction and patient-ventilator asynchrony detection.

Lung ultrasound monitoring, e.g., pleural effusion monitoring, atelectasis progression tracking, pneumonia progression tracking and lung sliding monitoring.

Hemodynamic monitoring, e.g., blood flow and plaque monitoring.

Fetal monitoring, e.g., fetal heart beat monitoring.

Liver monitoring, e.g., monitoring for liver disease.

Renal ultrasound, e.g., monitoring after transplantation, renal-blood flow monitoring

Bladder monitoring, e.g., bladder volume, obstruction, foley catheter placement or malfunctions, bladder stones.

Prostate enlargement monitoring

Cardiovascular home monitoring, e.g., electrocardiograph monitoring, arterial pressure monitoring.

Carotid artery narrowing monitoring, e.g., stroke monitoring, cardiac output monitoring, atrial fibrillation monitoring.

Heart muscle function and cardiac output in an acute care setting, e.g., monitoring after a heart attack.

Home ventilation monitoring to predict exacerbations.

Wearable ultrasound transducers may also be used for many other applications not mentioned here.

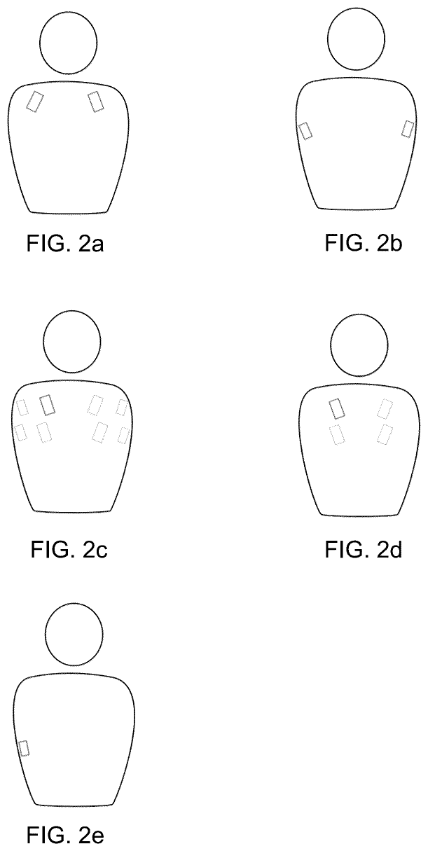

For illustrative purposes only, FIG. 2 displays exemplary placements of an ultrasound patch for various lung monitoring examinations:

FIG. 2a shows placements for examining and/or monitoring lung sliding (upper anterior zones);

FIG. 2b shows placements for examining and/or monitoring pleural effusion (lower lateral zones);

FIG. 2c shows placements for examining and/or monitoring pneumonia (risk zones or all zones);

FIG. 2d shows placements for examining and/or monitoring atelectasis (risk zones, only anterior); and

FIG. 2e shows placements for examining and/or monitoring diaphragm monitoring (lateral right, at base of diaphragm).

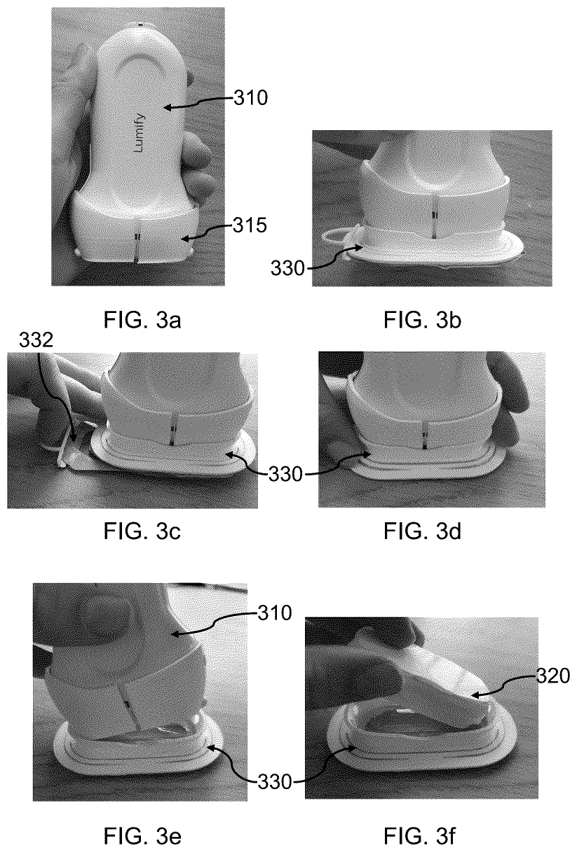

FIGS. 3a to 3f display an example of the method 1000 as executed in practice.

FIG. 3a displays a Philips Lumify ultrasound probe 310, wherein the first ultrasound transducer array is part of the ultrasound probe 310, connected to an adapter 315.

FIG. 3b displays the ultrasound probe 310 connected to the patch 330.

FIG. 3c shows how a liner 332 covering an adhesive layer below the patch 330 may be removed.

FIG. 3d shows how the patch may be connected with the surface of a subject by pushing down the adhesive layer onto the surface of the subject. In particular, the patch 330 has a spring like construction to enable movement of the adhesive layer towards the surface of the subject.

FIG. 3e displays the separation or removal of the ultrasound probe 310 from the patch 330.

FIG. 3f shows the connection of the second ultrasound transducer array 320 with the patch 330, here as part of a minimum functionality ultrasound probe, such that the combination of the patch and the second ultrasound transducer array is smaller in size and better suited as a wearable ultrasound patch. At the same time, the minimum functionality design enables increased up-time (i.e., the time of operation when not connected to a power supply) and reduces the overall financial cost of manufacturing.

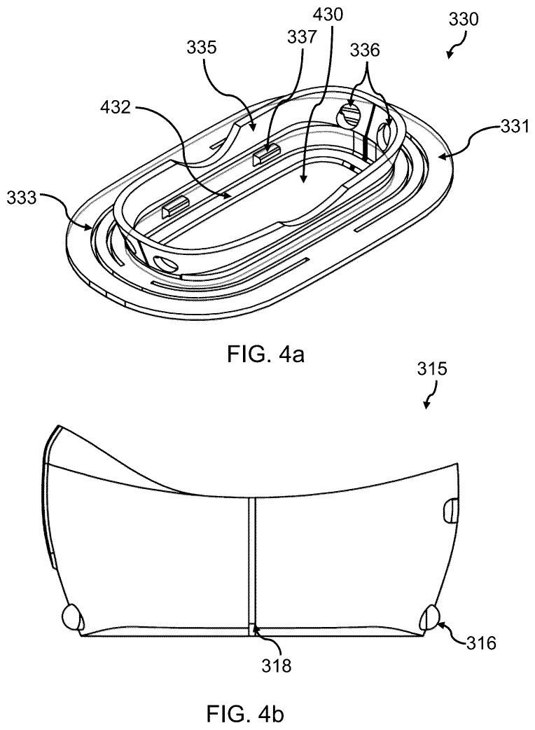

FIG. 4a shows a patch 330 for use in a wearable ultrasound patch according to an embodiment of the present invention. In particular, FIG. 4a shows a patch with:
a first connection mechanism to connect the patch with a surface of a subject; a second connection mechanism to connect the patch to a first ultrasound
transducer; and
a third connection mechanism to connect the patch to a second ultrasound transducer.

The first connection mechanism comprises a plate 331, wherein a first side (the lower side in FIG. 4a) is configured to be placed on the surface of a subject. The first side of the plate 331 may further comprise an adhesive layer enabling the connection between the patch and the surface of the subject. The adhesive layer may further be covered by a removable liner 332 (FIG. 3c displays the liner 332 being removed). The liner 332, is for example, configured to stop the first side of plate 331 from adhering to the surface of the subject while moving the first transducer array-patch combination according to step 1020 of method 1000. Once a physician wishes to connect the patch to the surface of a subject, the physician may remove the liner as shown in FIG. 3c thus connecting the patch to the surface of the subject. The plate 331 of the first connection mechanism further comprises various cut-outs 333 enabling a spring like behavior of the plate 331. This spring-like behavior is advantageous to compensate for skin movements of the subject that is being monitored. For example, tension and/or compression of the skin may occur while the subject is moving. In other words, the cut-outs 333 enable a more secure and comfortable adhesion with the subject.

It is noted that other first connection mechanisms are envisaged by the present invention such as vacuum options wherein a vacuum is generated between the patch and the surface of the subject to hold the patch at the target position. For example, a method to generate such a vacuum is to integrate one or multiple suction cups at the bottom of plate 331. Such a suction cup can have multiple form factors such as circular or it can have the form of plate 331 such that one suction cup covers the entire bottom surface of the plate. Additionally, a mechanism may be provided to generate vacuum in the suction cup. Such a mechanism may include a mechanism to increase the inner volume of the suction cup or to remove the air from the suction cup, e.g., by a piston or a miniature pump.

The second and third connection mechanisms shown in FIG. 4a are the same. However, this is not necessary and using different second and third connection mechanisms is entirely possible and envisaged by the present invention. The second connection mechanism in FIG. 4a (thus also the third connection mechanism) is configured to connect an ultrasound transducer array to the patch. In some embodiments, an adapter is connected to the second connection mechanism, wherein the adapter connects to an ultrasound transducer array. The connection mechanism comprises a cone-shaped opening 335 with cut-outs 336 and protrusions 337 to enable a secure connection (i.e., interlocking) between the ultrasound transducer array or an adapter and the patch.

The patch 330 may further comprise a cut-out or region of transparent or translucent material 430. This cut-out or region may be designed to hold a gel-pad. For example, a gel-pad may be inserted through the cone-shaped opening 335 and placed in the opening 430 while the edges of the gel-pad may be resting on surface 432. The gel-pad may be any gel-pad, but typically a solid gel-pad made out of mostly aqueous materials. In an example, the backside of the gel-pad, the side that is in contact with the first and/or second ultrasound transducer array may be slightly cone shaped where the highest point is in the center such that when connecting either of the first and second ultrasound transducer arrays the air is pressed outwards. As a result, no air bubbles should remain at the interface between the gel-pad and the ultrasound transducer surface.

FIG. 4b shows an adapter 315 to connect an ultrasound probe with the patch 330 of FIG. 4a according to an embodiment of the present invention.

In particular, the adapter 315 may be designed to bridge size and/or shape differences between an ultrasound transducer array 310 and the patch 330. In this particular embodiment, the adapter 315 is designed to fit the ultrasound transducer array 310 of the Philips Lumify hand-held ultrasound probe. However, other forms for the adapter 315, depending on the form and form factor of the first transducer array are also envisaged by the present invention.

The adapter 315 may further comprise protrusions 316 designed to fit into the cut-outs 336 of the patch enabling an interlocking between the patch and the adapter, thus enabling an interlocking between the patch and the ultrasound probe connected to the adapter.

The adapter 315 may further comprise a cut-out 318. This cut-out 318 may be used to increase flexibility of the adapter and reduce manufacturing tolerances, or may also be used to work together with the second connection mechanism by enabling pushing the protrusions 316 towards each other, thus making it easier to take the protrusions 316 out of the cut-outs 336 of the patch.

FIG. 4c shows an ultrasound transducer array 320 designed to form, when in combination with the patch 330 of FIG. 4a, a wearable ultrasound patch.

The ultrasound probe 320, similarly as the adapter 315, contains protrusions 326, designed to fit into the cut-outs 336 of the patch 330.

The ultrasound probe 320 may further comprise the second ultrasound transducer array, wherein the second ultrasound transducer array is configured for monitoring a particular feature of a subject as described above. Other forms for the ultrasound probe 320, different to the one depicted in FIG. 4c, wherein the form depends on the form and form factor of the second transducer array are also envisaged by the present invention. In an example, the second ultrasound transducer array may be connected to the patch 330 via an adapter similar to adapter 315.

The patch 330, the adapter 315 and the casing of the ultrasound transducer array 320 may be of any suitable material (e.g., metal, plastic, particularly polyethylene, combination of materials, etc.) and manufacturing method (e.g., 3D printed, casted, assembled from different parts, etc.).

While a specific adapter and second and third connection mechanisms have been described above, namely that of a clicking system, other alternatives are also envisaged by the present invention. For example, the first and second ultrasound transducer array may be connected to the patch 330 by the use of an adhesive. In an example, the first and second ultrasound transducer array may be connected to the patch via a pressure system. In particular, an operator may exert pressure on the first and/or second ultrasound transducer array while pushed against the surface of the subject, thus keeping the patch and the ultrasound transducer array together. In yet other examples, a magnetic system may be used wherein in magnets are integrated into the patch and the first and/or second ultrasound transducer array or the adapter 315, wherein the magnets are configured to keep the patch and the ultrasound transducer array connected via a magnetic force.

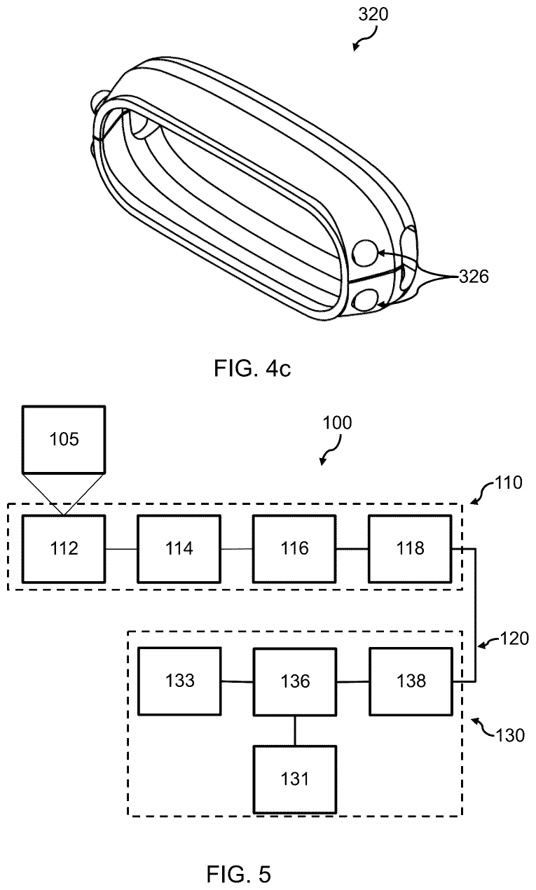

An exemplary ultrasound system according to the present disclosure is disclosed in FIG. 5. FIG. 5 shows a schematic diagram of an ultrasound imaging system 100. The system 100 includes an ultrasound imaging probe 110 in communication with a host 130 over a communication interface or link 120. The probe 110 may include an ultrasound transducer array 112, a beamformer 114, a processor 116, and a communication interface 118. The ultrasound transducer array 112 may be the first ultrasound transducer array and/or the second ultrasound transducer array. The host 130 may include a display 131, a processor 136, a communication interface 138, and a memory 133. The host 130 and/or the processor 136 of the host 130 may also be in communication with other types of systems or devices in replacement or addition to the here mentioned systems such as an external memory, external display, a subject tracking system, an inertial measurement unit, etc. It is understood that the beamformer may also be a microbeamformer. It is further understood that the components as shown here may also be configured in alternate arrangements. For example, the processor 116 and/or the beamformer 114 may be located outside of the probe 110 and/or the display 131 and/or memory 133 may be located outside of the host 130.

In some embodiments, the probe 110 is an ultrasound imaging device including a housing configured for handheld operation by a user. The ultrasound transducer array 112 can be configured to obtain ultrasound data while the user grasps the housing of the probe 110 such that the ultrasound transducer array 112 is positioned adjacent to or in contact with a patient's skin. The probe 110 is configured to obtain ultrasound data of anatomy within the patient's body while the probe 110 is positioned outside of the patient's body. In some embodiments, the probe 110 can be a patch-based external ultrasound probe. For example, the probe may be a hemodynamic patch.

The ultrasound transducer array 112 emits ultrasound signals towards an anatomical object 105 (e.g., a patient) and receives echo signals reflected from the object 105 back to the ultrasound transducer array 112. The ultrasound transducer array 112 can include any suitable number of acoustic elements, including one or more acoustic elements and/or a plurality of acoustic elements. In some instances, the ultrasound transducer array 112 includes a single acoustic element. In some instances, the ultrasound transducer array 112 may include an array of acoustic elements with any number of acoustic elements in any suitable configuration. For example, the ultrasound transducer array 112 can include between 1 acoustic element and 10000 acoustic elements, including values such as 2 acoustic elements, 4 acoustic elements, 36 acoustic elements, 64 acoustic elements, 128 acoustic elements, 500 acoustic elements, 812 acoustic elements, 1000 acoustic elements, 3000 acoustic elements, 8000 acoustic elements, and/or other values both larger and smaller. In some instances, the ultrasound transducer array 112 may include an array of acoustic elements with any number of acoustic elements in any suitable configuration, such as a linear array, a planar array, a curved array, a curvilinear array, a circumferential array, an annular array, a phased array, a matrix array, a one-dimensional (1D) array, a 1.x-dimensional array (e.g., a 1.5D array), or a two-dimensional (2D) array. The array of acoustic elements (e.g., one or more rows, one or more columns, and/or one or more orientations) can be uniformly or independently controlled and activated. The ultrasound transducer array 112 can be configured to obtain one-dimensional, two-dimensional, and/or three-dimensional images of the anatomical object 105. In some embodiments, the ultrasound transducer array 112 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof.

The beamformer 114 is coupled to the ultrasound transducer array 112. The beamformer 114 controls the ultrasound transducer array 112, for example, for transmission of the ultrasound signals and reception of the ultrasound echo signals. In some embodiments, the beamformer 114 may apply a time-delay to signals sent to individual acoustic transducers within an array in the ultrasound transducer array 112 such that an acoustic signal is steered in any suitable direction propagating away from the probe 110. The beamformer 114 may further provide image signals to the processor 116 based on the response of the received ultrasound echo signals. The beamformer 114 may include multiple stages of beamforming. The beamforming can reduce the number of signal lines for coupling to the processor 116. In some embodiments, the ultrasound transducer array 112 in combination with the beamformer 114 may be referred to as an ultrasound imaging component. The beamformer 114 may also comprise of one or multiple microbeamformers.

The processor 116 is coupled to the beamformer 114. The processor 116 may also be described as a processor circuit, which can include other components in communication with the processor 116, such as a memory, beamformer 114, communication interface 118, and/or other suitable components. The processor 116 is configured to process the beamformed image signals. For example, the processor 116 may perform filtering and/or quadrature demodulation to condition the image signals. The processor 116 and/or beamformer 114 can be configured to control the array 112 to obtain ultrasound data associated with the object 105.

The communication interface 118 is coupled to the processor 116. The communication interface 118 may include one or more transmitters, one or more receivers, one or more transceivers, and/or circuitry for transmitting and/or receiving communication signals. The communication interface 118 can include hardware components and/or software components implementing a particular communication protocol suitable for transporting signals over the communication link 120 to the host 130. The communication interface 118 can be referred to as a communication device or a communication interface module.

The communication link 120 may be any suitable communication link. For example, the communication link 120 may be a wired link, such as a universal serial bus (USB) link or an Ethernet link. Alternatively, the communication link 120 may be a wireless link, such as an ultra-wideband (UWB) link, an Institute of Electrical and Electronics Engineers (IEEE) 802.11 WiFi link, or a Bluetooth link.

At the host 130, the communication interface 138 may receive the image signals. The communication interface 138 may be substantially similar to the communication interface 118. The host 130 may be any suitable computing and display device, such as a workstation, a personal computer (PC), a laptop, a tablet, or a mobile phone.

The processor 136 is coupled to the communication interface 138. The processor 136 may also be described as a processor circuit, which can include other components in communication with the processor 136, such as the memory 133, the communication interface 138, and/or other suitable components. The processor 136 can be configured to generate image data from the image signals received from the probe 110. The processor 136 can apply advanced signal processing and/or image processing techniques to the image signals. An example of image processing includes conducting a pixel level analysis to evaluate whether there is a change in the color of a pixel, which may correspond to an edge of an object (e.g., the edge of an anatomical feature). In some embodiments, the processor 136 can form a three-dimensional (3D) volume image from the image data. In some embodiments, the processor 136 can perform real-time processing on the image data to provide a streaming video of ultrasound images of the object 105.

The memory 133 is coupled to the processor 136. The memory 133 can be configured to store patient information, measurements, data, or files relating to a patient's medical history, history of procedures performed, anatomical or biological features, characteristics, or medical conditions associated with a patient, computer readable instructions, such as code, software, or other application, as well as any other suitable information or data. The memory 133 may be located within the host 130. There may also be an additional external memory, or an external memory in replacement of memory 133. An external memory may be a cloud-based server or an external storage device, located outside of the host 130 and in communication with the host 130 and/or processor 136 of the host via a suitable communication link as disclosed with reference to communication link 120. Patient information may include measurements, data, files, other forms of medical history, such as but not limited to ultrasound images, ultrasound videos, and/or any imaging information relating to the patient's anatomy. The patient information may include parameters related to an imaging procedure such a probe position and/or orientation.

The display 131 is coupled to the processor 136. The display 131 may be a monitor or any suitable display or display device. The display 131 is configured to display the ultrasound images, image videos, and/or any imaging information of the object 105.

The system 100 may be used to assist a sonographer or operator in performing an ultrasound scan. The scan may be performed in a point-of-care setting. In some instances, the host 130 is a console or movable cart. In some instances, the host 130 may be a mobile device, such as a tablet, a mobile phone, or portable computer. In yet other examples the host is a server on a cloud and an external display connects to the host in the cloud. During an imaging procedure, the ultrasound system 100 can acquire an ultrasound image of a region of interest of a subject.

The use of wearable ultrasound patches for continuous (or regular) assessment of diaphragm thickness has several applications, such as ventilator induced diaphragm dysfunction and diaphragm atrophy detection (i.e., the monitoring of disturbed muscle contractility and gradual muscle thinning during mechanical ventilation, leading to poor prognosis, due to reduced weaning and extubation success and extended duration of mechanical ventilation); weaning prediction (i.e., studies have shown that extubation success can be predicted based on TFdi measurements during a spontaneous breathing trial); and patient-ventilator asynchrony detection (i.e., continuous diaphragm thickness measurements may be used to detect and quantify presence of patient-ventilator asynchronies such as trigger delays) (see, e.g., P. Tuinman et al. Respiratory muscle ultrasonography: methodology, basic and advanced principles and clinical applications in ICU and ED patients—a narrative review. Intensive Care Med. 2020).

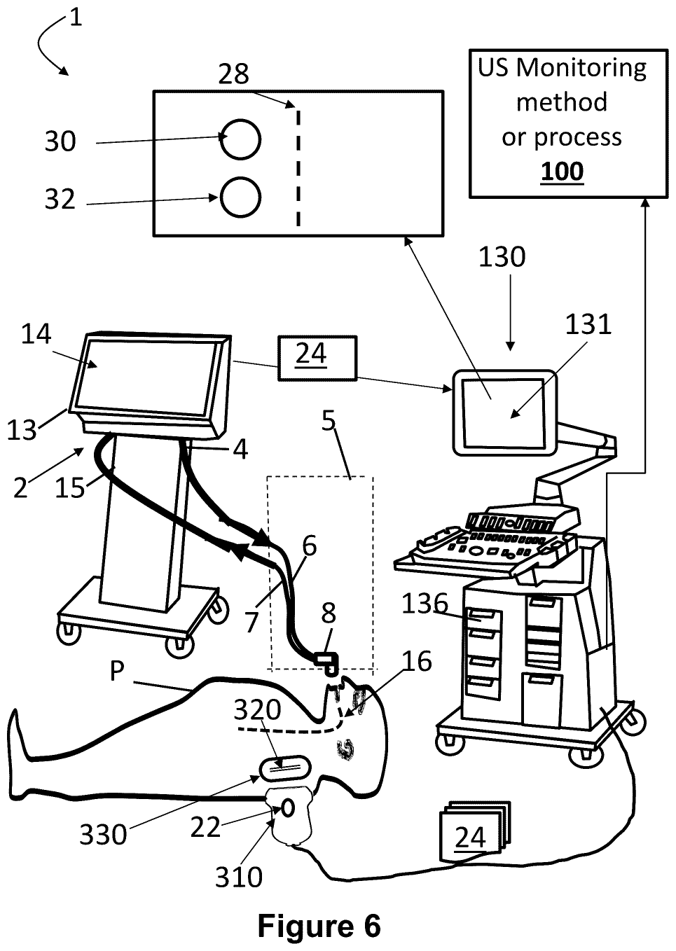

FIG. 6 shows another embodiment of an ultrasound imaging system 1 showing a patient P undergoing mechanical ventilation therapy. The ultrasound imaging system 1 generally includes an ultrasound patch 330 including at least one ultrasound transducer 320, a mechanical ventilator 2, and an electronic processing device (i.e., the host 130 which can include a computer) with a display 131 to display ultrasound images acquired by a handheld ultrasound probe 110. The host 130 is also wirelessly connected with the patch 330 and receives ultrasound data along a scanline acquired by the ultrasound probe 320. In addition, the host 130 can control both the ultrasound transducer 320 and the ultrasound imaging probe 310, for example, to change ultrasound patch settings of the ultrasound patch 330.

The mechanical ventilator 2 is connected with the host 130, and synchronized with the host 130. The host 130 receives ventilator data such as pressure and flow waveforms, and ventilator settings. For applications such as cardiac output monitoring, modalities such as an electro-cardiogram (ECG) may be connected to the host 130, in order to keep track of the phase of the cardiac cycle.

As described in more detail below, the host 130 is configured to, for example, display a virtual patch scanline in the acquired ultrasound images (i.e., to indicate the location of the patch scanline in the images); store one or more images; label ultrasound images with the phase of the breathing cycle, as determined from ventilator waveforms; accept user input to identify the anatomical structure (boundaries) of interest or alternatively, automatically identifying the anatomical structure of interest, determine the intersection between a virtual patch scanline and the anatomical structure boundaries and displaying markers at the intersections and calculating the distance between the markers; determine the angle between the virtual patch scanline and a line orthogonal to the anatomical structure; calculate optimal patch settings such as optimal focus depth; label patch data with the phase of the breathing cycle as determined from ventilator waveforms; determine the boundaries of the anatomical structure of interest in the patch data using information from the stored image(s) and calculating the distance between these boundaries; correct the calculated distance for the orientation of the anatomical structure with respect to the scanline, and display the values of the corrected results and storing and/or exporting the results for analysis.

As shown in FIG. 6, A mechanical ventilator 2 is configured to provide ventilation therapy to an associated patient P is shown. As shown in FIG. 6, the mechanical ventilator 2 includes an outlet 4 connectable with a patient breathing circuit 5 to deliver mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 6 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 6 shows the patient P already intubated. That is, FIG. 6 shows the patient after a tracheal intubation has been performed to insert the ETT 16 into the patient. However, to safely perform the tracheal intubation, the anesthesiologist or other qualified medical professional first performs an assessment of the patient P to select the ETT size of the ETT 16, and then inserts an ETT of the selected size into the patient P by a tracheal intubation procedure.

As previously described, an ultrasound patch 340 includes the patch 330 and the at least one transducer 320, and the ultrasound patch 340 is placed at a target position on a patient P. In some embodiments, a patch holder (i.e., the adapter 315) is configured for attachment to the patient P at the target position on the patient and configured to receive the patch 330. In some embodiments, the ultrasound imaging probe 310 is configured to acquire ultrasound images of the patient P. The ultrasound imaging probe 310 is positioned to acquire US data or US imaging data (i.e., US images) 24 of the diaphragm of the patient P. For example, the ultrasound patch 340 is configured to acquire US data of a diaphragm of the patient P, and more particularly US data related to a thickness of the diaphragm of a patient P during inspiration and expiration while the patient P undergoes mechanical ventilation therapy with the mechanical ventilator 2.

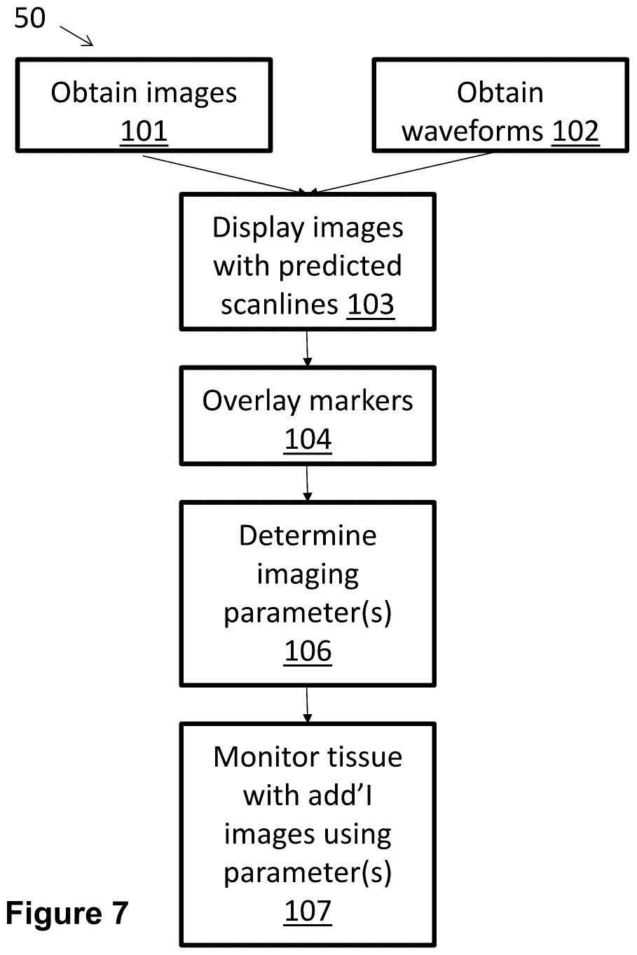

The host 130 also stores instructions executable by the electronic processor 136 to perform an ultrasound monitoring method or process 50. With reference to FIG. 7, and with continuing reference to FIG. 6, an illustrative embodiment of the ultrasound monitoring method 50 is diagrammatically shown as a flowchart.

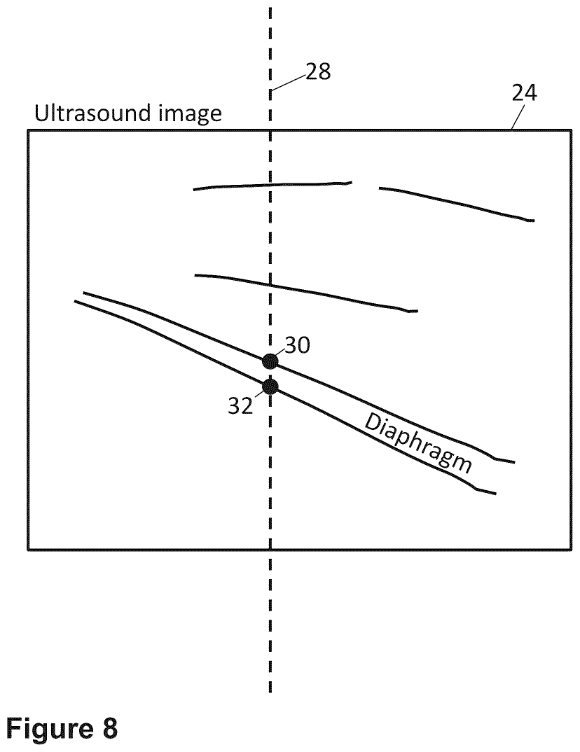

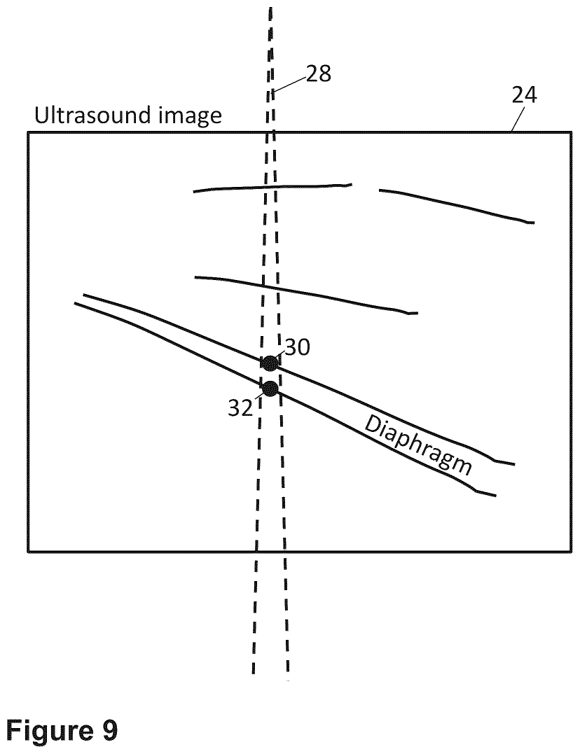

At an operation 101, the US images 24 of a target tissue (i.e., the diaphragm of the patient P) are acquired at a target position on the patient P using the ultrasound imaging probe 310, and transmitted to the host 130. In some embodiments, initial ultrasound images 24 of the patient P are acquired using the ultrasound imaging probe 310 at various locations around the target position, and transmitted to the host 130. In some examples, the display 131 can display the transmitted images 24 along with a virtual scanline (i.e., a vertical line mimicking a scanline acquired by the ultrasound transducer arrays 320 of the ultrasound patch 340) to indicate a predicted scanline location for the ultrasound patch 340 (which is not yet in place). This is illustrated in FIG. 8, which diagrammatically shows an ultrasound image 24 acquired by the ultrasound imaging probe 310 which depicts the edges of the thoracic diaphragm, and a superimposed predicted target scanline 28 indicating the predicted location of the scanline acquired by the ultrasound patch 340. In a variant embodiment shown in FIG. 9, the predicted target scanline 28 includes some width indicating uncertainty of its position. In illustrative FIG. 9, this uncertainty increases with increasing distance into the patient P (where increasing distance corresponds to downward in the ultrasound image 24 of FIG. 9), so that the predicted target scanline annotation 28 increases in width with increasing depth into the patient P. In some suitable implementations of this approach of FIG. 9, a (semi-transparent) area around the predicted ultrasound patch scanline can be color-coded to indicate a confidence level of the predicted ultrasound patch scanline location.

For some embodiments, the host 130 then determines an ultrasound probe position at which each initial ultrasound image 24 is acquired using ultrasound probe position tracking data received from an inertial measurement unit (IMU) 22 (i.e., an accelerometer) attached to the ultrasound probe 310. One of the initial ultrasound images that optimally images the target tissue of the patient is selected, and the target position on the patient P is determined as the ultrasound probe position at which the selected one of the initial ultrasound images is acquired.

The patch 330 is adhered to the patient P at the target position. The adhering of the patch 330 to the patient P on skin at the target position of the patient P includes removing a protection sheet from the patch 330 to expose adhesive of the patch 330. Ultrasound imaging can be performed with the ultrasound imaging probe 310 attached to the patch 330, and can then be detached from the patch 330.

At an operation 102, respiration data from the patient P is received by the host 130. The respiration data is time synchronized with the acquired ultrasound images 24 of the diaphragm of the patient P. The respiration data can be, for example, one or more ventilator waveforms 26 (e.g., pressure waveforms, flow waveforms, ventilator settings, and so forth) from the mechanical ventilator 2 transmitted to the host 130. At least one acquired ultrasound image 24 acquired at a target respiration phase is determined and selected to determine the target position of the patient P.

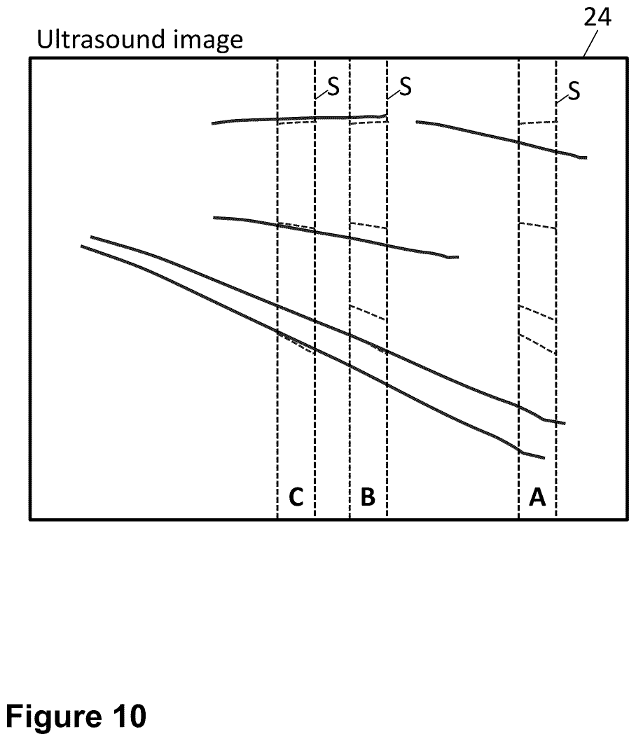

At an operation 103, the selected ultrasound image(s) 24 is displayed on the display device 131 of the host 130. The predicted target scanline annotation 28 is overlayed on the displayed image(s) 24. The predicted target scanline annotation 28 corresponds to a location of a predicted target scanline of the ultrasound patch 340 when the ultrasound patch 340 is placed at the target position on the patient P. The predicted target scanline 28 can be determined to indicate a position of an ultrasound path scanline of the ultrasound patch 340 in the ultrasound image(s) 24 when the ultrasound patch 340 is placed at the target position on the patient P. With reference to FIG. 10, in some embodiments the predicted target scanline annotation 28 is determined as a line or strip portion of the ultrasound image(s) 24 having maximum cross-correlation with an ultrasound scanline acquired by the ultrasound patch 340 placed at the target position on the patient P. FIG. 10 diagrammatically illustrates this approach by showing three positions A, B, and C of the ultrasound patch scanline S overlayed on an ultrasound image 24, and it is seen that at position C the ultrasound patch scanline S most closely matches the corresponding portion of the ultrasound image 24. Hence, position C will have the highest cross-correlation value. (Note, in diagrammatic FIG. 10 the patch scanlines S are shown using dashed lines to visually distinguish from the ultrasound image 24).

At an operation 104, first and second markers 30, 32 are overlayed on the displayed image(s) 24. The first and second markers 30, 32 can be, for example, circles that indicate boundaries of an image of the diaphragm in the displayed image(s) 24. FIGS. 8 and 9 diagrammatically illustrate examples of the diaphragm boundary markers 30 and 32 in the diagrammatically shown ultrasound image 24. Both the scanline annotation 28 and the first and second markers 30, 32 can be input by a clinician touching the display 131 (i.e., the display 131 is a touch screen) or other user input device (e.g., a mouse or keyboard). The markers 30, 32 can be an intersection of the predicted target scanline annotation 28 and the diaphragm (for example, a boundary of the diaphragm at the pleural membrane and the peritoneal membrane). The distance between the markers 30, 32 is determined and stored for some or all recorded images 24. The host 130 can implement an edge detection algorithm for precise alignment of the markers 30, 32 on the intersection using manually placed marker locations as a starting point. In the case multiple images 24 are stored, the markers 30, 32 are transferred (i.e., copied) from the displayed image to the other images (e.g., using motion tracking techniques such as block-matching by cross-correlation). In another example, a specific preset can be defined such as ‘costal diaphragm’ and selected by the user for automatic indication of the structure of interest. The intersection with the predicted target scanline annotation(s) 28 is then automatically or manually indicated.

The ultrasound imaging probe 310 is then removed, while the patch 330 position is maintained at the target position by removing the liner 332. To do so, in one approach the ultrasound imaging probe 310 is detached from the adapter 315, and the second ultrasound transducer array 320 is attached to the patch 330.

At an operation 106, at least one data acquisition parameter and/or data processing parameter (e.g., frequency, time-variable gain, steering angle, focus depth, aperture size and position, and so forth) is determined based on the displayed image(s) 24.

In some examples, when a data acquisition parameter is determined, additional images 24 of the diaphragm can be acquired using the ultrasound patch 340 with one or more settings of the ultrasound transducer 320 set to values equal to the at least one data acquisition parameter. The data acquisition or processing parameter is transferred from the host 130 to the ultrasound patch 340. For example, once the depth of the diaphragm is known, parameters such as optimal focus depth and gating window(s) can be determined, and the relevant ultrasound patch settings can be adapted accordingly. In addition, settings of the probe 310 (e.g., dynamic range (gain, compression) and time gain compensation) may be transferred to the ultrasound patch 340. Depending on the capabilities and complexity of the ultrasound patch 340, other setting parameters may include frequency, steering angle, aperture size and position (i.e., selection of active elements in the ultrasound transducer array 320).

At an operation 107, the diaphragm of the patient P is monitored by acquiring additional images 24 with the ultrasound probe 310 (or with measurements or scans acquired by the ultrasound patch 340) using the data acquisition parameter and/or the data processing parameter.

In the following, some illustrative examples of specific embodiments of the process of FIG. 7 or portions thereof are described.

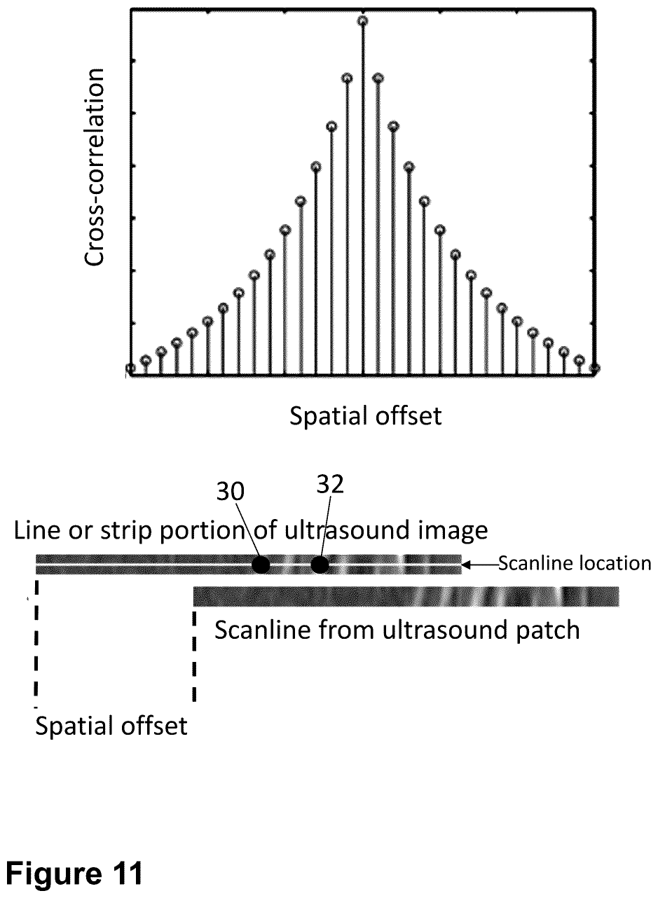

In some embodiments, boundaries of the diaphragm are determined, and the distance between these boundaries is calculated. In one example, the imaging data is converted to a series of 1-dimensional grey scale (brightness) images 24 each having the ultrasound patch scanlines S. Optionally, settings of the ultrasound imaging probe 310 such as dynamic range and time gain compensation may be used to enhance signal similarity between images generated by the first and second transducer arrays 310, 320. From the images 24 acquired by the ultrasound imaging probe 310, the scanline as indicated by the predicted target scanline annotation 28 is selected (see FIG. 8 or FIG. 9). Next, the cross-correlation between the ultrasound patch scanlines S in the first 1-D image and the predicted target scanline annotation 28 is calculated and the maximum value and location (i.e., lag) corresponding with the maximum value is determined. This can be done in two directions: perpendicular to the scanline as shown in FIG. 10; and parallel with the scanline as shown in FIG. 11. In the case of the parallel alignment of FIG. 11, for a specific line or strip portion of the ultrasound image 24. It is contemplated to iterate between the steps of FIG. 10 and FIG. 11 to optimize the location of the scanline in both orthogonal directions.

Since the dimensions of the anatomical structure of interest may change over time (e.g., the thickness of the diaphragm changes within a breathing cycle) a probe image 24 and an ultrasound patch scanlines S from the same phase of the breathing cycle is selected using the ventilator waveforms. Alternatively, the cross-correlation is calculated for all patch scanlines S (tᵢ from t₁ to t_{N}). Finally, the location and corresponding lag of the maximum cross-correlation value is determined to find the predicted target scanline annotation 28 (and relative shift) that correlates best with the actual ultrasound patch scanline S. Alternatively, the cross-correlation calculation is done for all ultrasound patch scanlines (ti from t1 to tN). Next, the location and corresponding lag of the maximum cross-correlation value is determined to find the predicted target scanline annotation 28 (and relative shift) that correlates best with the ultrasound patch scanlines S from the selected probe image 24.

Next, in the actual ultrasound patch scanline S, the location of the markers 30, 32 are determined (e.g., by transferring the location of the markers 30, 32 from the predicted target scanline annotation 28 to the actual ultrasound patch scanline S and searching for peaks in the actual ultrasound patch scanline S 1-D grey scale image close to these transferred marker locations). Finally, the distance between the marker locations 30, 32 in the ultrasound patch data is calculated. Optionally, several verification steps may be applied such as checking whether the maximum cross-correlation value exceeds a predetermined value and whether the distance between the peaks in the selected patch scanline does not deviate a predetermined value from the distance between markers in the selected image 24. In case one of the above-mentioned quality measures is not met, the cross-correlation may be performed on another probe image 24, or the user is asked to repeat the patch placement procedure.

In another example, reflections in the imaging data corresponding to the structure boundaries are determined. In the case of the diaphragm, the two strong echoes corresponding with the reflections from the pleural and peritoneal membranes can be determined (e.g., by peak detection) in the time window. These strong echoes should correspond to the location of the markers 30, 32 in both the predicted target scanline annotation 28 and the ultrasound patch scanlines S. It should be noted that the speed of sound that is used for the conversion from time delay to distance should be the same as is used in the image reconstruction. In a verification step the calculated range of distances from the patch data may be compared with the distance range as obtained from the probe images. Other methods such as pre-trained machine learning algorithms may be used to resolve the structure of interest in the patch data. Depending on the application the values of the corrected results are displayed, stored and/or exported for further analysis.

In the foregoing, the ultrasound patch 340 is assumed to acquire a one-dimensional scanline along a fixed direction. However, in some other examples, the ultrasound patch 340 can generate perpendicular as well as angulated scanlines, in combination with a 2D capable transducer. In this case, in the step 103 multiple predicted target scanline annotations 28 are displayed. The steps and principles to identify the anatomical structure of interest remains the same and is applied to all scanlines.

In other examples, the ultrasound imaging probe 310 and the ultrasound patch 340 are capable of generating electronically translatable 2D imaging planes. These may be cMUT based transducers. In this case, multiple images 24 with multiple predicted target scanline annotations 28 may be displayed. The steps and principles to identify the anatomical structure of interest remains the same and is applied to all scanlines.

In further examples, the ultrasound imaging probe 310 and the ultrasound patch 340 are 3D capable. Again, multiple images 24 with multiple predicted target scanlines annotations 28 may be displayed. The steps and principles to identify the anatomical structure of interest remains the same and is applied to all scanlines.

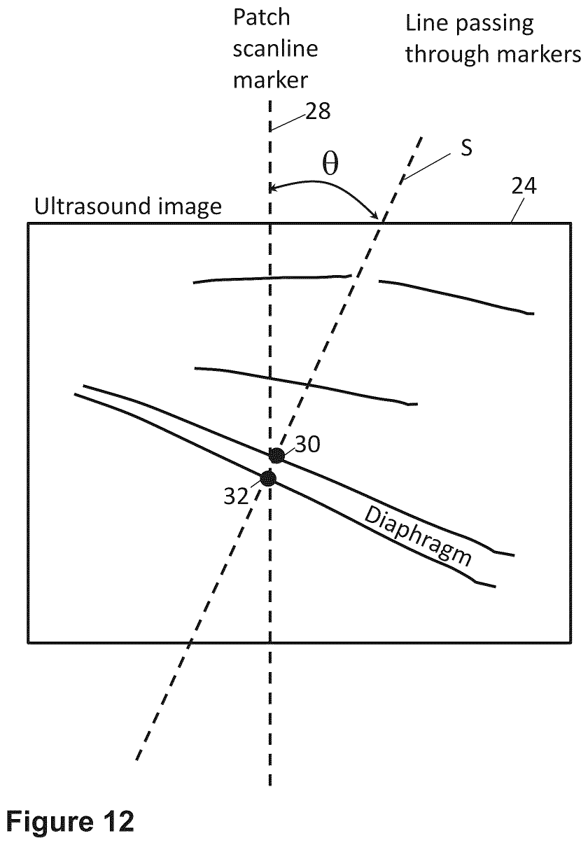

With reference to FIG. 12, in some embodiments, an orientation of the diaphragm with respect to the predicted (and thus also to the actual) target scanline annotations 28 can be determined from the images 24, and data derived from the images 24 (e.g., diaphragm thickness) can be corrected for angulation errors. To do so, as shown in FIG. 12 an angle θ between the predicted target scanline annotations 28 and a normal vector on the diaphragm surface can be determined. A diaphragm thickness is then determined from the additional scans or images 24 and a correction factor (i.e., the data acquisition parameter and/or data processing parameter) computed from the angle θ between the predicted target scanline annotations 28 and the diaphragm surface normal to determine the diaphragm thickness. In another example, the angle θ can be computed between a line orthogonal to the diaphragm (more particularly, the peritoneal membrane) and the scanline S. As an example, this is done by determining the angle θ between the scanline and a line orthogonal to the diaphragm. The peritoneal membrane can be detected using image analysis techniques such as Hough-transform and Radon-transform based line detection algorithms. The calculated thickness or distance of the diaphragm is corrected for the orientation of the peritoneal membrane with respect to the scanline. To this end, the thickness determined along the ultrasound patch scanline S is multiplied by a cosine of the angleθ resulting in the calculated thickness. The angle θ is preferably calculated in 3D (i.e., the diaphragm may also be inclined in the direction perpendicular to the image plane).

In another example, at least one phase of a breathing cycle of the patient P can be identified from the ventilator waveforms 26 and the acquired images 24. A position of the diaphragm can be determined from the identified phase(s) of the breathing cycle.

In some embodiments, a shift in a position of the patient P can be determined from the acquired image(s) 24. A position of the ultrasound patch 340 can be adjusted based on the determined position shift. For example, proning may cause the scan area to drift away from the region of interest, leading to invalid or unreliable ultrasound data. The second transducer 320 can be positioned to allow for small translations and tilt variations relative to the patch 330. After proning, the tilt and position of the second transducer 320 is manually or automatically adapted. In another example, the second transducer 320 can be electronically translatable or steerable, or one of the transducers of the second transducer array 320 can be selected as a “best positioned” transducer to determine an adjusted position of the patch 330. In other examples, a body position sensor (not shown) may be used to detect body repositioning events or body position (e.g., after proning). For example, based on this information ultrasound patch data may be labeled with a body position indicator such as to allow for comparing (e.g., diaphragm thickness data from same body positions and to exclude data from other body positions). In another example, based on the detected body position, an artificial intelligence (AI) model (not shown) may be used to compensate ultrasound-based measurements such as diaphragm thickness for body repositioning effects. For large drifts, a therapist may be notified for patch repositioning. In addition, the data may be labelled as invalid. If the probe 310 is used for a therapy such as a diaphragm training program, the therapy may be stopped.

In some embodiments, a patch placement method is performed without use of an adapter 315. First, the ultrasound imaging probe 310 is used to manually localize the desired anatomical feature and the corresponding desired ultrasound patch location, as may be done conventionally. The user maintains the ultrasound imaging probe 310 in a perpendicular position relative to the tissue surface. While scanning for the correct position, the ultrasound images 24 are stored for subsequent automatic processing using a suitable algorithm. Inertial measurement unit (IMU) sensor data from the sensor 22 may be used to measure the relative position of subsequent 2D acquisitions. In doing so, (quasi-) volumetric ultrasound data is acquired of the region surrounding the correct patch location. Optionally, to ease the ultrasound volume generation, the operator may be requested to perform a manual linear motion centered around the correct patch location in order to better generate the required (quasi-) volumetric ultrasound data.

Inside the acquired ultrasound volume surrounding the correct patch location, predicted or actual ultrasound patch scan lines perpendicular to the tissue surface may be reconstructed, each having along its length a distinct pattern of regions, varying in length, having higher or lower echogenicity. This distinct pattern is then used to compare the scan lines from the patch during patch placement.

Prior to patch placement, a connection is established between the ultrasound patch 340 and the host 130 that has access to the reconstructed scan line data from the ultrasound volume surrounding the correct patch location. The ultrasound patch 340 is set to localization mode, in which it acquires scan lines at short time intervals and transmits these scan lines to the host 130. The ultrasound patch 340 is subsequently coarsely placed onto the tissue surface sufficiently close to the correct patch location, in such a way, that the actual ultrasound patch scan lines from the patch fall inside the previously acquired ultrasound volume surrounding the correct patch location. The host 130 registers the actual ultrasound patch scan lines S with the ultrasound images 24 acquired with the probe 310. After registration, the actual position of the ultrasound patch 340 relative to the correction position is known. Since the position of the scan line relative to the correct patch location is known, directions may be given to the operator to expedite navigation to the correct position. Finally, an indication may be given to the operator that the correct position is reached, at which point the patch may be fixed in place, and utilization of the ultrasound patch 340 may start.

In some embodiments, ventilator waveforms 26 may be used to select ultrasound images and actual/predicted ultrasound patch scanlines from the same phase of the breathing cycle.

In some embodiments, the correct location may be found for an ultrasound patch 340 capable of generating perpendicular as well as angulated scan lines, in combination with a 2D capable transducer. Non-perpendicular scan lines from the ultrasound patch 340 and the acquired probe ultrasound volume images 24 may be compared for increased localization robustness. To ease the registration of the actual ultrasound patch scanline S with the ultrasound volume images 24, information from the ultrasound patch 340 about the angle of the scan line may be sent to the host 130.

In some embodiments, a probe capable of generating electronically translatable 2D imaging planes may be used to generate the 3D ultrasound volume needed for correct patch placement. This may be a capacitive micromachined ultrasonic transducer (cMUT) based probe. When using the probe to manually find the correct patch location a center plane of the probe may be used. Once the correct patch location is found, all other image planes inside the transducer field of view may be acquired automatically, from which the 3D ultrasound volume is constructed using a suitable algorithm. Since the relative position of the image planes is known, an IMU sensor may not be needed if the transducer is kept still during the parallel plane acquisitions.

In other embodiments, a 3D capable probe may be used to generate the 3D ultrasound volume directly. Since the volume is generated directly, an IMU sensor may not be needed if the transducer is kept still during the volume acquisition.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention may be implemented by means of hardware comprising several distinct elements, and/or by means of a suitably programmed processor. In the device claim enumerating several means, several of these means may be embodied by one and the same item of hardware. Measures recited in mutually different dependent claims may advantageously be used in combination.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform an ultrasound monitoring method, the ultrasound monitoring method comprising:
receiving at least one ultrasound image of a target tissue of a patient (P) acquired using an ultrasound imaging probe positioned at a target position on the patient;
determining at least one data acquisition and/or data processing parameter based on the at least one ultrasound image of the target tissue acquired using the ultrasound imaging probe; and
monitoring the target tissue of the patient including acquiring tissue data of the patient using an ultrasound patch comprising at least one ultrasound transducer placed at the target position on the patient and using the at least one data acquisition and/or data processing parameter.

2. The non-transitory computer readable medium of claim 1, wherein the ultrasound patch is configured to acquire the tissue data comprising ultrasound data collected along one or more ultrasound patch scanlines, and the ultrasound monitoring method further includes:
displaying the at least one ultrasound image on a display device; and
overlaying a predicted target scanline annotation on the displayed at least one ultrasound image;
wherein the predicted target scanline annotation indicates a predicted location of a scanline of the ultrasound patch when the ultrasound patch is placed at the target position on the patient.

3. The non-transitory computer readable medium of claim 1, wherein the target tissue of the patient is a diaphragm of the patient, the at least one ultrasound image include a time sequence of ultrasound images, and the ultrasound monitoring method further includes:
receiving respiration data from the patient time synchronized with the acquisition of the at least one ultrasound image of the diaphragm of the patient;
selecting, from the time sequence of ultrasound images and based on the respiration data, at least one target phase ultrasound image acquired at a target respiration phase;
displaying the at least one target phase ultrasound image on a display device; and
overlaying first and second markers on the displayed at least one acquired ultrasound image;
wherein the first and second markers indicate predicted boundaries of an image of the diaphragm in the displayed at least one target phase ultrasound image when the ultrasound patch is placed at the target position on the patient.

4. The non-transitory computer readable medium of claim 3, wherein the ultrasound monitoring method further includes:
determining a predicted target phase scanline annotation indicating a position of an ultrasound scanline of the ultrasound patch in the at least one target phase ultrasound image when the ultrasound patch is placed at the target position on the patient.

5. The non-transitory computer readable medium of claim 4, the predicted target phase scanline annotation comprising a line or strip portion of the at least one target phase ultrasound image having maximum cross-correlation with an ultrasound scanline acquired by the ultrasound patch placed at the target position on the patient.

6. The non-transitory computer readable medium of claim 4, wherein the ultrasound monitoring method further includes:
determining an angle between the predicted target scanline annotation and a line orthogonal to the diaphragm surface; and
wherein the monitoring of the target tissue includes determining a diaphragm thickness metric from the tissue data of the diaphragm of the patient acquired using the ultrasound patch, and the at least one data acquisition and/or data processing parameter comprises a correction factor computed from the angle between the scanline annotation and the first and second markers, the correction factor being applied when determining the diaphragm thickness metric.

7. The non-transitory computer readable medium of claim 3, wherein the received respiration data comprises one or more ventilator waveforms received from a mechanical ventilator providing mechanical ventilation therapy to the patient (P).

8. The non-transitory computer readable medium of claim 1, wherein the ultrasound monitoring method further comprises:
receiving initial ultrasound images of the patient (P) acquired using the ultrasound imaging probe, wherein the initial ultrasound images are acquired at a plurality of different positions of the ultrasound imaging probe on the patient;
determining an ultrasound imaging probe position at which each initial ultrasound image is acquired using ultrasound imaging probe position tracking data received from an inertial measurement unit (IMU) attached to the ultrasound imaging probe;
selecting one of the initial ultrasound images that optimally images the target tissue of the patient; and
determining the target position on the patient as the ultrasound imaging probe position at which the selected one of the initial ultrasound images is acquired.

9. The non-transitory computer readable medium of claim 1, wherein the determined at least one data acquisition and/or data processing parameter includes at least one data acquisition parameter, and the ultrasound monitoring method includes:
acquiring the tissue data of the target tissue of the patient using the ultrasound patch with at least one setting of the ultrasound patch set equal to the determined at least one data acquisition parameter.

10. The non-transitory computer readable medium of claim 9, wherein the ultrasound monitoring method further comprises:
electronically transferring at least one data acquisition parameter from the at least one electronic processor to the ultrasound patch.

11. An ultrasound imaging method, comprising:
performing ultrasound imaging using an ultrasound imaging probe to identify a target position on a patient (P) at which the ultrasound imaging probe acquires images of a diaphragm of the patient;
placing an ultrasound patch comprising at least one ultrasound transducer at the target position on the patient;
determining at least one data acquisition and/or data processing parameter based on at least one ultrasound image of the diaphragm acquired by the ultrasound imaging probe during the ultrasound imaging; and
acquiring target tissue data of the target tissue of the patient using the ultrasound patch placed at the target position on the patient and using the at least one data acquisition and/or data processing parameter.

12. The method of claim 11, further comprising:
adhering a patch holder to the patient at the target position on the patient;
wherein the placing of the ultrasound patch at the target position on the patient includes placing the ultrasound patch in the patch holder adhered to the patient.

13. The method of claim 12, further wherein the ultrasound imaging is performed with the ultrasound imaging probe attached to the patch holder, the adhering of the patch holder to the patient at the target position of the patient includes removing a protection sheet from the patch holder to expose adhesive of the patch holder, and the method further includes
detaching the ultrasound imaging probe from the patch holder adhered to the skin of the patient.

14. The method of claim 11, further including:
determining a shift in a position of the patient (P); and
adjusting a position of the ultrasound patch based on the determined shift.

15. An ultrasound imaging system, comprising:
at least one electronic processor programmed to:
receive ultrasound images of a target tissue of a patient (P) acquired using an ultrasound imaging probe positioned at a target position on the patient;
determine at least one data acquisition and/or data processing parameter based on at least one ultrasound image of the target tissue acquired by the ultrasound imaging probe during the ultrasound imaging; and
monitor the target tissue of the patient including acquiring tissue data of the target tissue of the patient using an ultrasound patch comprising at least one ultrasound transducer placed at the target position on the patient and using the at least one data acquisition and/or data processing parameter.

16. The system of claim 15, further including:
an ultrasound patch comprising at least one ultrasound transducer placed at the target position on a patient.

17. The system of claim 15, further including:
the ultrasound imaging probe.

18. The system of claim 17, further including:
a patch holder configured for attachment to the patient (P) at the target position on the patient and configured to receive the ultrasound probe.

19. The system of claim 15, wherein the target tissue is a thoracic diaphragm.

20. The system of claim 15, further including:
a mechanical ventilator providing mechanical ventilation therapy to the patient (P);
wherein the at least one electronic processor is further programmed to:
receive one or more ventilator waveforms from the mechanical ventilator;
identify at least one phase of a breathing cycle of the patient from the ventilator waveforms and the acquired ultrasound images;
determine a position of the target tissue of the patient from the identified at least one phase of the breathing cycle.
